(19) United States
(12) Patent Application Publication
(10) Pub. No.: US 2024/0052281 A1
(43) Pub. Date: Feb. 15, 2024

(54) AIR BUBBLE REMOVAL DEVICE FOR REMOVING AIR BUBBLES FROM MEDIUM AND MEDIUM CIRCULATING CELL CULTIVATION SYSTEM INCLUDING THE SAME

(71) Applicant: AMOGREENTECH CO., LTD., Gimpo-si, Gyeonggi-do (KR)

(72) Inventors: Seon Ho JANG, Gimpo-si, Gyeonggi-do (KR); Seoung Hoon LEE, Gimpo-si, Gyeonggi-do (KR); In Yong SEO, Gimpo-si, Gyeonggi-do (KR); Chan KIM, Gimpo-si, Gyeonggi-do (KR); Kyoung Ku HAN, Gimpo-si, Gyeonggi-do (KR); Jae Yun KIM, Gimpo-si, Gyeonggi-do (KR)

(73) Assignee: AMOGREENTECH CO., LTD., Gimpo-si, Gyeonggi-do (KR)

(21) Appl. No.: 18/259,571

(22) PCT Filed: Dec. 29, 2021
(86) PCT No.: PCT/KR2021/020167

§ 371 Date: Jun. 27, 2023

(30) Foreign Application Priority Data
Dec. 30, 2020 (KR) 10-2020-0187016
Dec. 29, 2021 (KR) 10-2021-0190962

(51) Int. Cl. C12M 1/21; C12M 1/34

(52) U.S. Cl. CPC C12M 1/21; C12M 1/34; B01D 19/02

(57) ABSTRACT

An air bubble removal device for removing air bubbles from a medium including a housing including an inner space for storing a predetermined amount of the medium, an introduction port through which the medium is introduced from the outside into the inner space, and a discharge port through which the medium in a state in which air bubbles have been removed is discharged from the inner space to the outside; and a support part coupled to the housing so as to maintain the housing in a state spaced a predetermined height from a bottom surface.

### CROSS-REFERENCE TO RELATED APPLICATIONS

This application is the national phase entry of International Application No. PCT/KR2021/020167, filed on Dec. 29, 2021, which is based upon and claims priority to and the benefit of Korean Patent Application No. 10-2020-0187016, filed on Dec. 30, 2020, and Korean Patent Application No. 10-2021-0190962, filed on Dec. 29, 2021, the disclosures of which are incorporated herein by reference in their entireties.

### TECHNICAL FIELD

The present invention relates to an air bubble removal device for removing air bubbles from a medium and a medium circulating cell cultivation system including the same.

### BACKGROUND

Cell culture refers to culturing and propagating tissue pieces by supplying nutrients to the tissue pieces that are removed from a subject of multicellular organisms in a vessel.

Animal cells derived from human or animal tissue may be cultured while being suspended in a medium or attached to a support. Mainly, blood cell-derived cells (including hematopoietic stem cells) are suspension cells, and cells derived from tissues such as skin, liver or lungs, and embryonic stem cells, mesenchymal stem cells or the like are adherent cells.

Suspension cells can be proliferated alone in a state where cells are suspended in a medium, but adherent cells can proliferate only in a state of being attached to the surface of a support.

Accordingly, in order for the adherent cells to be smoothly cultured, it is important that the cells to be cultured are stably attached to the surface of a support.

The seeding process of attaching these adherent cells to the surface of a support is performed by injecting a medium containing the cells to be cultured into the inside of a vessel in which the support is disposed. That is, the cultured cells included in the medium adhere to the surface of the support when the medium comes into contact with the surface of the support.

However, when air bubbles are included in the medium that is supplied from the outside during the seeding process, the air bubbles contained in the medium prevent the cells from being stably attached to the surface of the support.

Accordingly, if the cells are cultured in a state where the cells are not stably attached to the surface of the support, there is a problem in that the cells cannot be stably cultured.

Moreover, in the medium circulating cell cultivation system that circulates the culture medium that is required for cell culture from a medium supply device to a cell cultivation device, when the culture medium containing air bubbles is supplied to the cell cultivation device, the cells attached to the support are not smoothly supplied with nutrients contained in the medium due to the air bubbles.

Accordingly, there is a problem in that cells that are not smoothly supplied with nutrients from the culture medium are not smoothly cultured.

Therefore, air bubbles contained in the medium need to be removed not only in the seeding process of attaching cells to the surface of the support, but also in the process of culturing the cells.

### SUMMARY OF THE INVENTION

The present invention has been devised in view of the above points, and is directed to providing an air bubble removing device for removing bubbles from a medium that is capable of supplying the medium in a state where air bubbles have been removed to the side of a support of a cell cultivation device, and a medium circulating cell cultivation system including the same.

In order to solve the above-described problems, the present invention provides an air bubble removal device for removing air bubbles from a medium which is a device for removing air bubbles from a medium, including: a housing which includes an inner space for storing a predetermined amount of the medium, an introduction port through which the medium is introduced from the outside into the inner space, and a discharge port through which the medium in a state where air bubbles have been removed is discharged from the inner space to the outside; and a support part which is coupled to the housing so as to maintain the housing in a state of being spaced by a predetermined height from a bottom surface, wherein the inner space includes a first space which is filled with a predetermined amount of the medium and a second space which is positioned above the first space and not filled with the medium, and wherein the first space and the second space are distinguished through the water level of the medium that is introduced into the inner space from the outside through the introduction port.

In addition, the introduction port may be provided in the housing to be located at a higher position than the discharge port, and the discharge port may be provided in the housing to be located at a position communicating with the first space, and the introduction port may be provided in the housing to be located at a position communicating with the second space.

In addition, the second space may be an air bubble separation space in which air bubbles are separated as the medium flowing into the inner space through the introduction port falls into the first space.

In addition, the discharge port may be provided in the housing to be located on a bottom surface of the inner space.

In addition, the air bubble removal device may further include a venting port which is provided in the housing to communicate with the second space.

In this case, the air bubble removal device may further include a pressure adjusting unit which is coupled to the venting port so as to control the amount of the medium stored in the inner space by adjusting the inner pressure of the inner space.

In addition, the housing may include a box-shaped housing body having the inner space and a cover member which is detachably coupled to the housing body so as to cover an open upper portion of the inner space.

In addition, the air bubble removal device may further include a sensing part including a sensor for detecting a state of the medium that is stored in a predetermined amount in the inner space.

In addition, the sensor may include at least one of a PH sensor for detecting the PH of the medium and a temperature sensor for measuring the temperature of the medium.

In addition, the sensor may be arranged such that a part of the length including one end is submerged in the medium that is filled in the first space.

In addition, the sensing part may be detachably coupled to one side of the housing.

In addition, the medium may be a seeding medium including cells to be cultured or a culture medium including nutrients.

Meanwhile, the present invention provides a medium circulating cell cultivation system, including a cell cultivation device in which at least one support to which cells to be cultured are attached is installed; a medium supply device in which a predetermined amount of culture medium for culturing the cells is stored therein; a cell supply source in which a predetermined amount of seeding medium including cells to be attached to the support is stored; an air bubble removal device for removing bubbles from the culture medium introduced from the medium supply device or the seeding medium introduced from the cell supply source, and then supplying the same to the side of the cell cultivation device; and a pump for circulating the culture medium, wherein the air bubble removal device is the above-described air bubble removal device.

According to the present invention, the seeding medium or culture medium may be supplied to a support included in the cell culture device in a state where air bubbles are removed. Through this, if the air bubble removal device for removing air bubbles from a medium according to the present invention is used, cells can be stably attached to the surface of the support, and the cells to be cultured can be smoothly supplied with nutrients from the medium such that it is possible to enable stable cell culture.

### BRIEF DESCRIPTION OF THE DRAWINGS

FIG. 1 is a schematic diagram showing the air bubble removal device for removing bubbles from a medium according to one embodiment of the present invention.

FIG. 2 is a diagram showing a state in which the main components are separated from FIG. 1.

FIG. 3 is a cutaway view of the housing of FIG. 1.

FIG. 4 is a view showing a state in which the medium is filled in the inner space in FIG. 3.

FIG. 5 is a schematic diagram showing a medium circulating cell cultivation system to which the air bubble removal device for removing bubbles from a medium according to one embodiment of the present invention is applied.

### DETAIL DESCRIPTION

Hereinafter, with reference to the accompanying drawings, the exemplary embodiments of the present invention will be described in detail so that those of ordinary skill in the art can easily practice the present invention. The present invention may be embodied in many different forms and is not limited to the exemplary embodiments described herein. In order to clearly describe the present invention in the drawings, parts that are irrelevant to the description are omitted, and the same reference numerals are assigned to the same or similar elements throughout the specification.

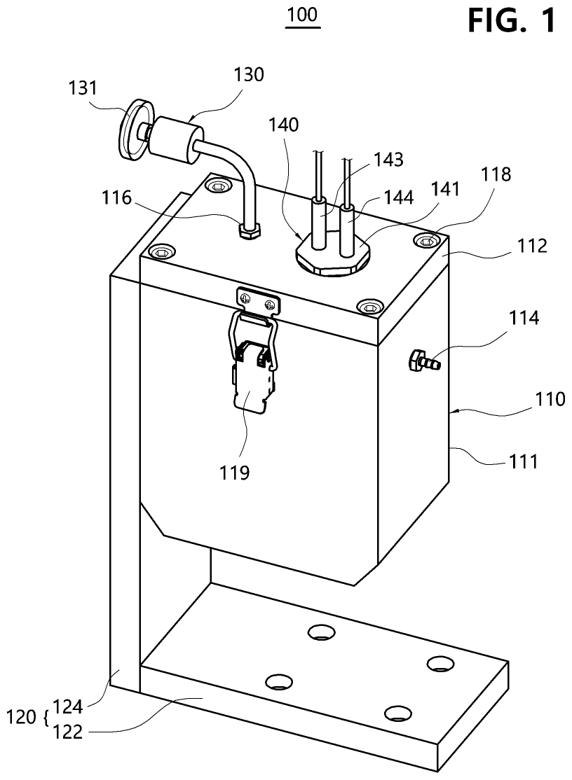

As illustrated in FIG. 1, the air bubble removal device 100 (hereinafter, referred to as ‘air bubble removal device’) for removing bubbles from a medium according to one embodiment of the present invention may include a housing 110 and a support part 120, and the medium introduced to the inside may be discharged to the outside in a state where air bubbles are removed.

Herein, the medium may be a seeding medium containing the cells to be cultured, or may be a culture medium containing nutrients required for cell culture.

That is, the seeding medium may be a medium in which the cells to be cultured are included, and the culture medium may be a medium in which the cells to be cultured are not included while containing nutrients necessary for culturing the cells.

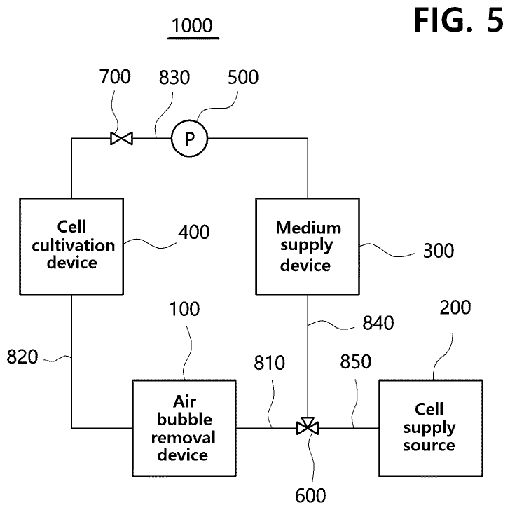

For example, the air bubble removal device 100 according to one embodiment of the present invention may be applied to a medium circulating cell cultivation system 1000 in which a medium is circulated as illustrated in FIG. 5.

In this case, the seeding medium containing the cells to be cultured may be supplied from a cell supply source 200 to the side of a cell cultivation device 400, and the cells included in the seeding medium may be attached to the surface of a support (not illustrated) that is provided in the cell cultivation device 400.

In addition, the culture medium containing nutrients required for cell culture may be sequentially circulated through the cell cultivation device 400, the air bubble removal device 100 and the medium supply device 300 by driving a pump 500.

That is, the air bubble removal device 100 removes air bubbles from the culture medium introduced from the medium supply device 300 or the seeding medium introduced from the cell supply source 200, and then, the culture medium or seeding medium from which air bubbles have been removed may be supplied to the side of the cell cultivation device 400.

Through this, the cells included in the seeding medium may be stably attached to the surface of a support (not illustrated) provided in the cell cultivation device 400, and the cells attached to the support may be cultured smoothly through nutrients that are supplied from the culture medium.

To this end, the housing 110 may include an internal space 113 having a predetermined volume such that a predetermined amount of the medium supplied from the outside can be stored, and the housing 110 may include an introduction port 114 for introducing the medium into the inner space 113 from the outside, and a discharge port 115 for discharging the medium stored in the inner space 113 to the outside.

In this case, the introduction port 114 may be connected to the medium supply device 300 or the cell supply source 200 through a connection pipe 810, and the discharge port 115 may be connected to the medium supply device 300 through a connection pipe 820.

Accordingly, the culture medium introduced from the medium supply device 300 through the introduction port 114 and the connection pipe 810 or the seeding medium introduced from the cell supply source 200 may be stored in the inner space 113 at a predetermined amount, and the medium stored in the inner space 113 may be discharged to the outside through the discharge port 115.

For example, the medium discharged through the discharge port 115 may move along the connection tube 820 to the cell cultivation device 400.

As a specific example, the housing 110 may include a box-shaped housing body 111 having the inner space 113 and a cover member 112 which is detachably coupled to one side of the housing body 111 so as to cover an open upper portion of the inner space 113.

In this case, the introduction port 114 may be provided in the housing body 111 to communicate with the inner space 113, and the discharge port 115 may be provided in the housing body 111 to communicate with the inner space 113.

In addition, the cover member 112 may be detachably coupled to the housing body 111 through at least one fastening member 118, 119.

For example, the fastening members 118, 119 may include at least one of a first fastening member 118 and a second fastening member 119, and the first fastening member 118 may be a known bolt member, and the second fastening member 119 may be a known toggle clamp.

As a non-limiting example, the toggle clamp may be a commercial name Cicada fastener.

Accordingly, the cover member 112 may be coupled to the housing body 111 through at least one of the first fastening member 118 and the second fastening member 119 so as to seal an open upper portion of the inner space 113.

Meanwhile, the medium introduced into the inner space 113 in the air bubble removing device 100 according to one embodiment of the present invention may be discharged to the outside through the discharge port 115 after the bubbles are removed.

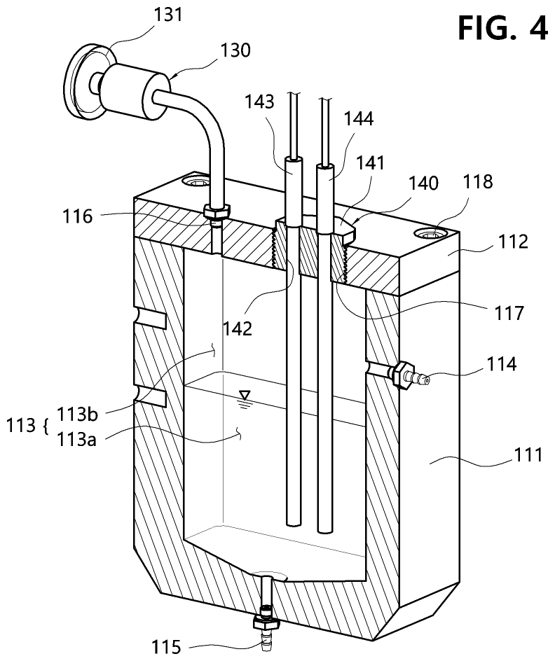

To this end, as illustrated in FIG. 4, the inner space 113 may be divided into a first space 113a that is filled with the medium and a second space 113b that is not filled with the medium, and the first space 113a and the second space 113b may be distinguished through the water level of the medium introduced into the inner space 113, and the second space 113b may be formed to be located on the upper side of the first space 113a.

In addition, the introduction port 114 may be provided in the housing 110 to be located at a higher position than the discharge port 115.

That is, the discharge port 115 may be provided in the housing 110 so as to communicate with the first space 113a that is formed on the lower side of the inner space 113, and the introduction port 114 may be provided in the housing 110 so as to communicate with the second space 113b that is located on the upper side of the internal space 113.

For example, the discharge port 115 may be provided in the housing 110 so as to communicate with the first space 113a while being positioned on a bottom surface of the inner space 113.

Accordingly, the medium introduced into the inner space 113 from the outside through the introduction port 114 may move to the first space 113a through the second space 113b, and the medium stored in the first space 113a may be discharged to the outside through the discharge port 115.

In this case, the second space 113b may serve as an air bubble separation space for separating air bubbles from the medium flowing into the inner space 113 through the introduction port 114.

That is, the medium introduced into the inner space 113 from the outside through the introduction port 114 may fall along the second space 113b such that air bubbles may be separated, and the medium in which the air bubbles have been separated in the second space 113b may move to the first space 113a.

Through this, the medium introduced into the inner space 113 can move to the first space 113a after air bubbles are removed from the second space 113b, and the medium filled in the first space 113a may be discharged to the outside in a state where air bubbles are removed through the discharge port 115.

For this reason, when the discharge port 115 is connected to the introduction port of the cell cultivation device 400, the medium supplied to the cell cultivation device 400 may be a medium in which air bubbles have been completely removed.

Through this, when the air bubble removal device 100 according to one embodiment of the present invention is used, the medium in a state in which the air bubbles have been removed can be supplied to the side of the cell cultivation device 400, and thus, the cells included in the seeding medium may be stably attached to the surface of a support.

In addition, when the culture medium in a state in which the air bubbles have been removed is supplied to the side of the cell cultivation device 400, the cells attached to the surface of a support can receive nutrients from the medium while receiving minimal stress.

In this case, the air bubble removal device 100 according to one embodiment of the present invention may adjust the amount of the medium stored in the inner space 113 by adjusting the internal pressure of the inner space 113.

To this end, the air bubble removal device 100 may include a venting port 116 provided in the housing 110 so as to communicate with the inner space 113, and the venting port 116 may be connected to a pressure adjusting unit 130.

For example, the venting port 116 may be provided in the cover member 112, and the venting port 116 may communicate with the second space 113b.

In this case, the pressure adjusting unit 130 may include a vent filter 131, and may serve as a venting unit for discharging air bubbles that are separated from the medium and filled in the second space 113b to the outside.

Accordingly, the air bubble removing device 100 according to one embodiment of the present invention can adjust the water level of the medium flowing into the inner space 113 through the opening and closing of the pressure adjusting unit 130, and the inner space 113 may be divided into the first space 113a and the second space 113b through the water level of the medium.

In addition, the water level of the medium introduced into the inner space 113 may be changed or maintained through the opening and closing of the pressure adjusting unit 130.

Through this, the water level of the medium introduced into the inner space 113 may be adjusted through the pressure adjusting unit 130 to be maintained at a position lower than the position of the introduction port 114.

Accordingly, the medium introduced into the inner space 113 through the introduction port 114 may always move to the first space 113a via the second space 113b, and at the introduction port 114, the medium moving to the first space 113a may be a medium in which air bubbles have been separated in the second space 113b.

For this reason, as described above, the medium discharged to the outside through the discharge port 115 may always be in a state where air bubbles have been removed.

The support part 120 may support the housing 110 while being spaced apart from the bottom surface by a predetermined height.

For example, the support part 120 may include a bottom plate 122 and a side plate 124 extending at a predetermined height from the bottom plate 122, and one side of the housing 110 may be coupled to the side plate 124.

In this case, the discharge port 115 may be disposed to be positioned on the lower side of the housing 110 while the housing 110 is coupled to the support part 120.

That is, the housing 110 may be coupled to the support part 120 such that the introduction port 114 is located at a higher position than the discharge port 115, and although the discharge port 115 is formed on the lower side of the housing 110, it may be disposed to maintain a state of being spaced apart from the bottom plate 122 by a predetermined height.

Accordingly, when a predetermined amount of medium flows into the inner space 113 in a state where the housing 110 is coupled to the support part 120, the discharge port 115 may communicate with the first space 113a, and the introduction port 114 may communicate with the second space 113b.

However, the shape of the support part 120 is not limited thereto, and as long as it has a shape that can space apart the housing 110 from the bottom surface by a predetermined height, it may be appropriately changed according to design conditions.

Meanwhile, the air bubble removal device 100 according to one embodiment of the present invention may further include a sensing part 140 including sensors 143, 144 for detecting the state of the medium stored in the inner space 113.

That is, the sensors 143, 144 may include at least one of a PH sensor 143 for detecting the PH of the medium and a temperature sensor 144 for measuring the temperature of the medium.

In this case, as illustrated in FIG. 4, the sensors 143, 144 may be provided in the housing 110 such that at least a part thereof is in contact with the medium stored in the inner space 113.

That is, the sensors 143, 144 may be disposed such that a part of the length including one end is submerged in the medium filled in the first space 113a.

Accordingly, the air bubble removal device 100 according to one embodiment of the present invention may monitor the state of the medium before being supplied to the cell cultivation device 400 through the sensors 143, 144 such that it is possible to maintain the medium supplied to the cell cultivation device 400 through the discharge port 115 in an optimal state.

In this case, the sensing part 140 may be detachably coupled to one side of the housing 110.

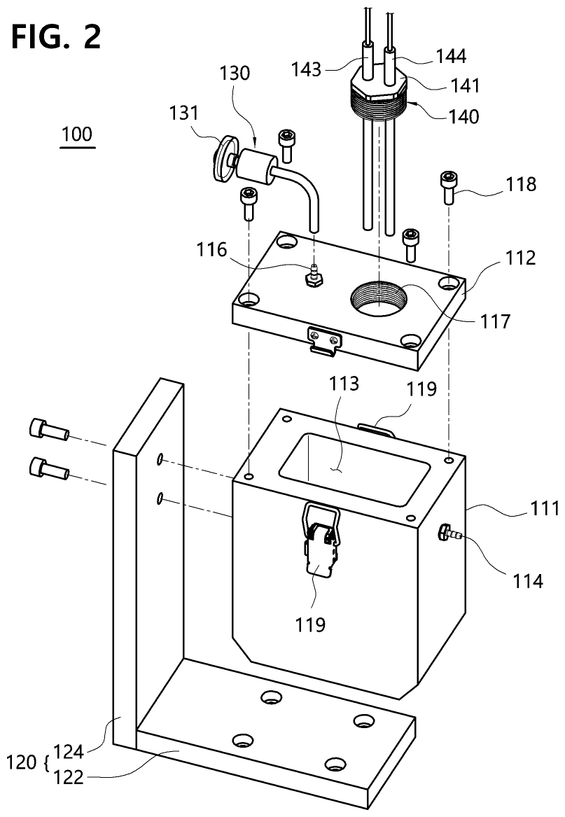

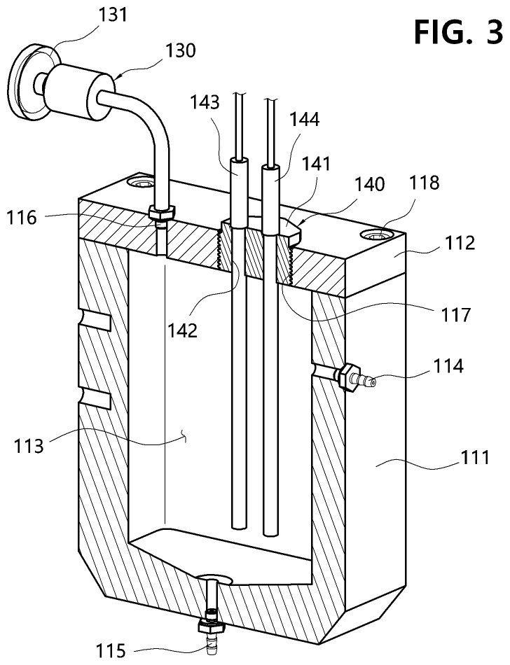

For example, the sensing part 140 may include a stopper 141 that is detachably coupled to the housing 110 as illustrated in FIGS. 2 and 3, and the stopper 141 may include at least one coupling hole 142 for fixing the sensors 143, 144.

Herein, the stopper 141 may be detachably coupled to the fastening hole 117 that is formed through the cover member 112, and the sensors 143, 144 may be detachably coupled to the coupling hole 142.

Moreover, the sensors 143, 144 may be connected to a controller (not illustrated) through a cable.

Accordingly, the user can monitor and manage the status of the medium based on the information measured through the sensors 143, 144.

However, the type of the sensor is not limited thereto, and various known sensors may be applied as long as it can measure information on the status of the medium required for cell culture.

Meanwhile, the air bubble removal device 100 according to one embodiment of the present invention may be applied to the medium circulating cell cultivation system 1000 as described above.

For example, as illustrated in FIG. 5, the medium circulating cell cultivation system 1000 according to one embodiment of the present invention may include a cell cultivation device 400, a medium supply device 300, a cell supply source 200, an air bubble removal device 100 and a pump 500.

Herein, in the cell cultivation device 400, at least one support (not illustrated) to which cells to be cultured are attached may be installed therein, and the medium supply device 300 may store a predetermined amount of culture medium required for cell culture, and the cell supply source 200 may store a predetermined amount of the seeding medium required for cell culture.

Further, in the cell supply source 200, the seeding medium stored therein may be moved to the side of the air bubble removal device 100 through the injection of a gas having a predetermined carbon dioxide concentration, and the culture medium may be maintained at a constant PH concentration that is required for cell culture in the medium supply device 300.

In this case, the cell cultivation device 400, the medium supply device 300, the cell supply source 200, the air bubble removal device 100 and the pump 500 may be interconnected via a plurality of connection pipes 810, 820, 830, 840, 850.

In addition, the seeding medium stored in the cell supply source 200 or the culture medium stored in the medium supply device 300 may be selectively supplied to the air bubble removal device 100 through the opening and closing of at least one valve 600, 700.

For example, the culture medium stored in the medium supply device 300 may be sequentially circulated in the medium supply device 300, the air bubble removal device 100 and the cell cultivation device 400 through changing the opening/closing state of the at least one valve 600, 700 and driving the pump 500.

In this case, the culture medium may be supplied to the air bubble removal device 100 from the medium supply device 300, and the culture medium introduced into the air bubble removal device 100 may be supplied to the side of the cell cultivation device 400 after air bubbles have been removed in the air bubble removal device 100. Moreover, the culture medium moved to the cell cultivation device 400 may be recovered to the medium supply device 300 and supplied again to the air bubble removal device 100 in a state of being restored to a constant PH concentration required for cell culture.

In contrast, the seeding medium stored in the cell supply source 200 may be supplied to the air bubble removal device 100 through the opening and closing of the at least one valve 600, 700.

In this case, the seeding medium may be moved to the air bubble removal device 100 by an injection pressure provided from the cell supply source 200 and then supplied to the side of the cell cultivation device 400.

Accordingly, the seeding medium may be supplied to the side of the cell cultivation device 400 in a state where the air bubbles have been removed in the air bubble removal device 100.

Through this, the cells included in the seeding medium can be smoothly attached to the side of a support that is accommodated in the cell cultivation device 400, and the seeding medium moved to the cell cultivation device 400 may stay in the cell cultivation device 400 without moving to the side of the medium supply device 300.

For this reason, the cells included in the seeding medium supplied from the air bubble removal device 100 to the cell cultivation device 400 may be smoothly attached to the surface of the support in the cell cultivation device 400.

In the drawings and description, the above-described bubble removal device 100 has been illustrated and described as being applied to a circulation system in which the medium is circulated, but the present invention is not limited thereto, and it may be applied to a stationary cell cultivation system in which the medium is not circulated. For example, the above-described bubble removal device 100 may be applied to a process of seeding cells or a process of first injecting a medium in a stationary cell cultivation system.

Moreover, the cells cultured in the cell cultivation device 400 can be utilized as cells for producing a large amount of artificial nanovesicles. That is, the cells cultured in the cell cultivation device 400 may be converted into artificial nanovesicles through cell disruption.

Although one embodiment of the present invention has been described above, the spirit of the present invention is not limited to the exemplary embodiments presented herein, and those skilled in the art who understand the spirit of the present invention may easily suggest other exemplary embodiments by modifying, changing, deleting or adding components within the scope of the same spirit, but this will also fall within the scope of the present invention.

1. An air bubble removal device for removing air bubbles from a medium which is a device for removing air bubbles from a medium, comprising:
a housing which includes an inner space for storing a predetermined amount of the medium, an introduction port through which the medium is introduced from the outside into the inner space, and a discharge port through which the medium in a state where air bubbles have been removed is discharged from the inner space to the outside; and
a support part which is coupled to the housing so as to maintain the housing in a state of being spaced by a predetermined height from a bottom surface,
wherein the inner space includes a first space which is filled with a predetermined amount of the medium and a second space which is positioned above the first space and not filled with the medium, and
wherein the first space and the second space are distinguished through the water level of the medium that is introduced into the inner space from the outside through the introduction port.

2. The air bubble removal device of claim 1, wherein the introduction port is provided in the housing to be located at a higher position than the discharge port,
wherein the discharge port is provided in the housing to be located at a position communicating with the first space, and
wherein the introduction port is provided in the housing to be located at a position communicating with the second space.

3. The air bubble removal device of claim 1, wherein the second space is an air bubble separation space in which air bubbles are separated as the medium flowing into the inner space through the introduction port falls into the first space.

4. The air bubble removal device of claim 1, wherein the discharge port is provided in the housing to be located on a bottom surface of the inner space.

5. The air bubble removal device of claim 1, wherein the air bubble removal device further comprises:
a venting port which is provided in the housing to communicate with the second space.

6. The air bubble removal device of claim 5, wherein the air bubble removal device further comprises:
a pressure adjusting unit which is coupled to the venting port so as to control the amount of the medium stored in the inner space by adjusting the inner pressure of the inner space.

7. The air bubble removal device of claim 1, wherein the housing includes a housing body having the inner space and a cover member which is detachably coupled to the housing body so as to cover an open upper portion of the inner space.

8. The air bubble removal device of claim 1, wherein the air bubble removal device further comprises:
a sensing part including a sensor for detecting a state of the medium that is stored in a predetermined amount in the inner space.

9. The air bubble removal device of claim 8, wherein the sensor includes at least one of a PH sensor for detecting the PH of the medium and a temperature sensor for measuring the temperature of the medium.

10. The air bubble removal device of claim 8, wherein the sensor is arranged such that a part of the length including one end is submerged in the medium that is filled in the first space.

11. The air bubble removal device of claim 8, wherein the sensing part is detachably coupled to one side of the housing.

12. The air bubble removal device of claim 1, wherein the medium is a seeding medium including cells to be cultured or a culture medium including nutrients.

13. A medium circulating cell cultivation system, comprising:
a cell cultivation device in which at least one support to which cells to be cultured are attached is installed;
a medium supply device in which a predetermined amount of culture medium for culturing the cells is stored therein;
a cell supply source in which a predetermined amount of seeding medium including cells to be attached to the support is stored;
an air bubble removal device for removing air bubbles from the culture medium introduced from the medium supply device or the seeding medium introduced from the cell supply source, and then supplying the same to the side of the cell cultivation device; and
a pump for circulating the culture medium,
wherein the air bubble removal device comprises:
a housing which includes an inner space for storing a predetermined amount of the medium, an introduction port through which the medium is introduced from the outside into the inner space, and a discharge port through which the medium in a state where air bubbles have been removed is discharged from the inner space to the outside; and
a support part which is coupled to the housing so as to maintain the housing in a state of being spaced by a predetermined height from a bottom surface,
wherein the inner space includes a first space which is filled with a predetermined amount of the medium and a second space which is positioned above the first space and not filled with the medium, and
wherein the first space and the second space are distinguished through the water level of the medium that is introduced into the inner space from the outside through the introduction port.
